(19) United States
(12) Patent Application Publication
(10) Pub. No.: US 2026/0258191 A1
(43) Pub. Date: Sep. 3, 2026

(54) USE OF HYDROXY SUBSTITUTED URONES AS CURE ACCELERATORS

(71) Applicant: HEXCEL COMPOSITES LIMITED, Duxford (GB)

(72) Inventor: Christopher MASON, Newmarket (GB)

(73) Assignee: HEXCEL COMPOSITES LIMITED, Duxford (GB)

(21) Appl. No.: 19/165,254

(22) PCT Filed: Mar. 11, 2024
(86) PCT No.: PCT/EP2024/056422

§ 371 Date: Sep. 15, 2025

(30) Foreign Application Priority Data
Mar. 17, 2023 (GB) 2303897.9

(51) Int. Cl. C08G 59/68; C07D 241/04; C08J 5/04

(52) U.S. Cl. CPC C08G 59/686; C07D 241/04; C08J 5/04; C08J 2363/00

(57) ABSTRACT

Urone compounds containing at least 2 orthohydroxyphenyl urea units where the units are coupled via a divalent or multivalent bridging group which may be heterointerrupted are used as cure accelerators for thermosetting resin systems particularly fibre filled epoxy resin.

The present invention relates to derivatives of urea, their use as curative accelerators for resin systems particularly epoxy resins, epoxy resin formulations containing the derivatives of urea and prepregs and moulded articles employing such epoxy resin formulations the preferred use is as a cure rate accelerator. Accordingly the invention provides urea based compounds, a curative system, a resin formulation, a cured resin, a use, a composite and a moulding material. The invention is also concerned with prepregs comprising fibrous material impregnated with curable resin.

A curative is a compound which is adapted to initiate or advance a polymerisation reaction of a polymerisable resin. An accelerator is a compound which enhances the polymerisation reaction (or “curing”) caused by a curative. This invention is particularly concerned with cure accelerators.

Urea derivatives, sometimes known as urones are known as is their use as curatives and cure accelerators for resin systems, particularly epoxy resins. For example, N,N¹ (Dimethyl) urones of the general formula

R—(NH—CO—N(CH³)2)n

are proposed as curatives for epoxy resin systems in U.S. Pat. No. 9,663,609. The use of these urones is said to enable an improved controlled curing of epoxy resin compositions in particular for the production of mouldings having a relatively large thickness which have previously been difficult to produce due to the large amount of heat generated during the curing of thick layers of epoxy resin.

The preferred urones of U.S. Pat. No. 9,663,609 are unsubstituted, halo substituted or alkyl substituted radical of formula

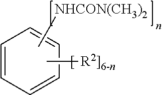

and

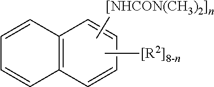

and various useful urones are listed.

Conventionally, a fiber-reinforced composite material composed of reinforcing fibers such as carbon fibers or glass fibers and a thermosetting resin such as an epoxy resin or a phenolic resin has been applied to many fields such as aerospace, automobiles, rail cars, marine vessels, civil engineering and construction, wind energy components and sporting goods. These composite materials are lightweight yet are excellent in mechanical properties such as strength and stiffness, heat resistance and corrosion resistance. Particularly, in applications requiring high performance, a fiber-reinforced composite material using continuous reinforcing fibers is used. As the reinforcing fibers, carbon fibers which are excellent in specific strength and specific modulus are used, and as a matrix resin, a thermosetting resin, particularly, an epoxy resin having adhesiveness particularly to carbon fibers, heat resistance, elastic modulus, and chemical resistance and having lowest curing shrinkage is often used. In recent years, required properties of the fiber-reinforced composite material have become more demanding. Particularly, when the fiber-reinforced composite material is applied to aerospace applications and structural materials of vehicles or the like, where it is required to sufficiently maintain physical properties even under high temperature and/or high humidity conditions.

As a hardener for epoxy resin, aromatic amine compounds, acid anhydrides and phenol novolac compounds are often used in aerospace applications requiring heat resistance.

However, these hardeners tend to require long heating times and high curing temperatures of around 180° C. during molding to effect cure of the resin. When reactivity of the epoxy resin composition is low, molding takes a long time, resulting in an increase in energy cost at molding. Therefore, a technology that enables curing of the epoxy resin composition to provide a moulding having the desired properties in a shorter time has been desired.

The curing of epoxy resins is usually an exothermic reaction and it is important that the reaction is controlled to avoid the development of excess temperatures that can degrade the epoxy material and can cause stress and deformation such as cracking in articles and components created from the epoxy containing formulation. There is however an ongoing need to decrease the time required for the cure and at the same time to increase the Tg of cured epoxy resins particularly to provide sufficient strength to the articles and components made from the resin formulation particularly when the resins are being used in resin impregnated fibre reinforcements sometimes known as prepregs that are used to produce larger and larger articles and particularly thicker articles made from stacks of prepregs such as stacks of more than 40, sometimes more than 60 up to 80 prepregs. These stacks of prepregs are often employed in the production of components for the aerospace and wind turbine industries where a prepreg or a stack of prepregs comprising a fibrous reinforcement impregnated with a curable resin ready for curing within a mould or vacuum bag is employed. To date a higher Tg has been obtained using higher curing temperatures and/or longer curing cycles and employing hardeners such as dicyandiamide which require higher curing temperatures; however, this increases the risk of high temperature degradation of the resin.

It has been proposed that the use of urones as curatives can enable the production of cured resins with higher Tgs to be obtained using lower cure temperatures and shorter cure cycles.

Derivatives of urea are known as is their use as curatives for epoxy resins and such derivatives are sometimes known as urones. U.S. Pat. No. 4,404,356 relates to hydroxy phenyl ureas of formula

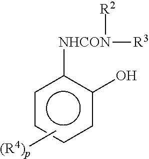

and to their use as accelerators for heat curing of epoxy resins and also to their use as primary curing agents for epoxy resins.

U.S. Pat. No. 9,663,609 relates to the use of bis or multifunctional N,N′-(Dimethyl) urones and a method for curing epoxy resin compositions using such urones. The use of these materials is said to provide a method for the controlled curing of epoxy resins particularly for solid components having large layer thickness of epoxy resins to avoid internal stresses or other thermal damage in mouldings or components that are produced therefrom. The bis- or multi-functional N,N′-(dimethyl) urones are of the general formula

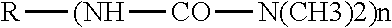

where R is a linear or branched aliphatic radical or an unsubstituted, halo substituted and/or alkyl substituted aromatic radical and n is a number from 2 to 20.

Currently the curatives that are used particularly those that are used in aerospace applications are aromatic amine compounds and anhydrides. However these systems typically require long cure cycle times at high temperatures which can typically be as high as 180°. Accordingly it would be beneficial to reduce the cycle time for the cure of the epoxy resin without any significant reduction in the service temperature (Tg) of the cured epoxy resin and other performance characteristics. This would enable production rates to be increased and the energy required for cure to be reduced.

In our PCT application published as WO 2022/034201 we describe bisfunctional orthohydroxy aromatic urones and their use as curatives for epoxy resins, whereby the resin can be cured at lower temperatures, such as an externally applied temperature from 80° C. to 150° C. to produce a cured epoxy resin having a higher Tg at complete cure than is produced when using the non-hydroxy substituted aromatic bisfunctional urones such as those described in U.S. Pat. No. 9,663,609 and the hydroxyl phenyl ureas of U.S. Pat. No. 4,404,356. The urones of WO 2022/034201 may also be used as accelerators for other epoxy curatives when the curing temperature may be higher for example up to 180° C.

WO 2022/034201 describes urones of the formulae selected from

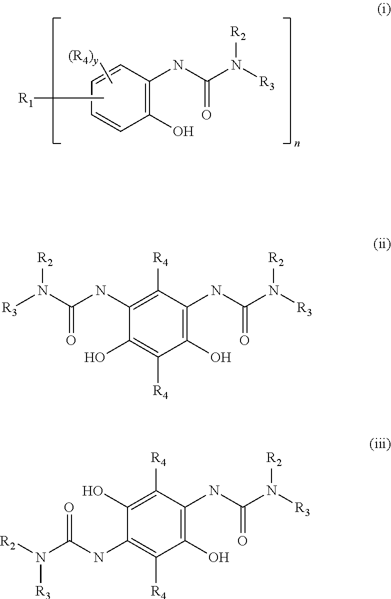

Where R₁ is selected from S, SO2, SO, P, PX, N, NH, NX, P═O(OX or OH), C═O, a linear or branched aliphatic radical (substituted or unsubstituted), or an unsubstituted, halo substituted and/or alkyl substituted aromatic radical and n is a number from 2 to 20.

Where X is a linear or branched aliphatic radical (substituted or unsubstituted), or an unsubstituted, halo substituted and/or alkyl substituted aromatic radical R₂ and R₃ at each occurrence, are independently of one another, selected from an alkyl, cycloalkyl, alkenyl, cycloalkenyl, or aralkyl group, which may be substituted by a halogen atom (preferably a chlorine atom) or by a hydroxyl or cyano group, with the proviso that R may alternatively denote a hydrogen atom, or R₂ and R₃ together with the indicated attached nitrogen atom denote a heterocyclic ring containing 3 to 5 carbon atoms, and optionally one oxygen atom.

R₄ at each occurrence, independently of one or another, is selected from H, NH₂, NO₂, nitrile, a halogen, linear or branched aliphatic radical, or an unsubstituted, halo substituted and/or alkyl substituted aromatic radical, and Y=1 to 3.

The compounds which may be used as curatives or accelerators for other curatives or they can perform both of these functions particularly with epoxy resins and WO 2022/034201 also describes an epoxy resin formulations containing one or more of these compounds optionally together with one or more other curatives as well as mouldings produced therefrom.

Where thick moulded structures are being produced such as in the aerospace and wind energy industries aromatic amines and anhydrides are employed as curatives and these systems require long cure cycle times at relatively high temperatures (such as around 180° C.). It would therefore be desirable to reduce the cycle time. However, it is also important that a resin system of epoxy resin and curative does not cure in a way that the curing reaction does not progress to an extent that reduces its workability of the uncured material at lower temperatures as this makes storage and transportation of the uncured system. The ability of a formulation to remain uncured at a certain temperature is known as its latency and one would not want to significantly reduce the latency in order to reduce the cure cycle time. Latency is also important to ensure good pot life at the elevated temperatures that can be used to manufacture prepregs.

We have now found if two or more ortho hydroxy phenyl based urones such as those described in of WO 2022/034201 are coupled via a bridging group containing two or more atoms the compounds are useful cure accelerators for resin systems such as epoxy resins and their use can enable shorter resin cure times and the production of mouldings with improved (higher) glass transition temperatures (Tg). The use of these urones as cure accelerators has also been found to extend the latency of the epoxy resin formulations with which they are used relative to formulations containing the dimethylamine blocked urones.

The invention therefore provides an accelerator for the curing of resins comprising a urone compound containing at least 2 orthohydroxyphenyl urea units wherein the urea units are coupled via a divalent or multivalent bridging group consisting of a linear branched or cyclo aliphatic hydrocarbon radical which is optionally hetero interrupted.

Preferred compounds according to this invention are selected from one or more compounds of the following formula

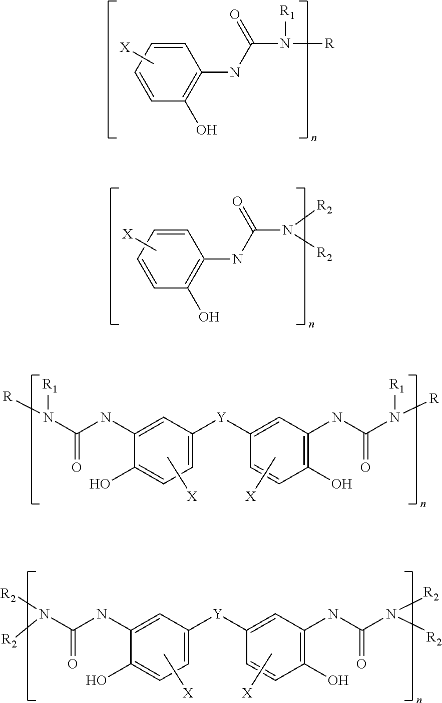

Where R is a divalent or multivalent linear or branched aliphatic hydrocarbon radical (substituted or unsubstituted), a divalent or multivalent hetero interrupted aliphatic hydrocarbon radical (substituted or unsubstituted), a divalent or multivalent aromatic hydrocarbon interrupted aliphatic hydrocarbon radical (substituted or unsubstituted).

Where R₁ is at each occurrence, independently of one or another, selected from H, linear or branched monovalent aliphatic radical (substituted or unsubstituted), or an unsubstituted, halo substituted and/or alkyl substituted monovalent aromatic radical.

Where both R₂ groups together forms a divalent or multivalent organic cycloaliphatic hydrocarbon radical (substituted or unsubstituted), a divalent or multivalent hetero interrupted cycloaliphatic hydrocarbon radical (substituted or unsubstituted), a divalent or multivalent aromatic hydrocarbon interrupted cycloaliphatic hydrocarbon radical (substituted or unsubstituted) Where Y is S, SO2, SO, PR₂, NR₂, P═O(OX or OH), C═O, a linear or branched aliphatic radical (substituted or unsubstituted), or an unsubstituted, halo substituted and/or alkyl substituted aromatic radical.

Where n is at least 2 Where X is at each occurrence, independently of one or another, selected from H, OH, NH₂, NO₂, nitrile, a halogen, a substituted or unsubstituted linear or branched aliphatic radical, and a substituted or unsubstituted aromatic radical.

The bridging group Y, is substituted with further orthohydroxyphenylurone functionalities units, to form a tri or tetravalent bridging unit, forming a network type oligo or poly-meric urone structure.

Where the urones of this invention are used as the primary or sole curative for the resin we prefer to use from 2 to 20 wt % of the urone based on the weight of the resin more preferably 5 to 15 wt %. Although their preferred use is as accelerators for other curatives.

The use of the urones of this invention as cure accelerators has the additional benefit that it provides latency at elevated temperatures which means that a prepreg can be obtained more easily and at the same time can accelerate the curing of the prepreg. Furthermore the presence of the urone in the uncured thermosetting resin systems has produced an uncured material containing the urone which can be stored for several weeks without significant cure (known as the latency or outlife of the system) and which can later be cured to produce a cured resin having a desirable Tg. This is particularly useful for applications which involve production of articles from prepregs. This is particularly useful when the resin is to be used to make components for the aerospace and wind energy industries particularly when the components are thick and made from a stack of several layers of the prepreg. Typically, at least 40 layers and sometimes 60 or more layers. The use of the urones of this invention also allows improved control of the heat generated during the curing reaction to reduce the risk of reaction run away which can cause degradation of the resin and damage to the cured article.

Examples of curatives whose performance can be accelerated by the compounds of this invention are primary or secondary amines. The amines may be aliphatic, cycloaliphatic, aromatic, or aromatic structures having one or more amino moieties.

The difference in the performance of the urones used in this invention rather than the previous tertiary amine functionalised urones such as those described in U.S. Pat. No. 9,663,609 is thought to be because the cure acceleration mechanism of the polymerisation of epoxy resins is due to the dissociation of the urone within the epoxy resin to form an isocyanate and an amine. The amine then reacts sequentially with two epoxy groups to form a quaternary amine and an alkoxide anion which propagates through the epoxy resin to effect cure. With the previous urones such as those containing dimethylamine groups the quaternary amine is formed immediately upon dissociation of the urone and so the polymerisation is dependent purely on the temperature at which dissociation takes place.

However we believe that due to the bulkier multifunctional groups in the bridging group of the urones used in this invention the initiation of the propagation of the polymerisation requires a higher temperature than that required to effect the dissociation of the urone.

The urones of this invention act as accelerators for amine curatives particularly aromatic amine curatives which cure at higher temperatures (such as 170° C. or higher) and this allows more of the epoxy amine addition to occur before the formation of the quaternary amine which leads to a higher Tg of the fully cured resin.

Exemplary amine curing agents whose performance can be accelerated by the urones of this invention include ethylenediamine, diethylenediamine, diethylenetriamine, triethylenetetramine, propylene diamine, tetraethylenepentamine, hexaethyleneheptamine, hexamethylenediamine, cyanoguanidine, 2-methyl-1,5-pentamethylene-diamine, 4,7,10-trioxatridecan-1,13-diamine, aminoethylpiperazine, and the like. Other exemplary curing agents include dicyanopolyamides, most preferably (DICY). 4,4′-diaminodiphenyl sulfone (4,4′-DDS) or 3,3′-diaminodiphenyl sulfone (3,3′-DDS) can also be beneficially employed as a latent amine curing agent, as well as mixtures of DICY and DDS. Dihydrazides such and ADH, IDH and polyamines such as Ancamine 2441 and BF3-MEA complexes such as Anchor 1040 (Air Products) are also suitable as a latent curing agent.

In some embodiments, the amine curing agent is a polyether amine having one or more amine moieties, including those polyether amines that can be derived from polypropylene oxide or polyethylene oxide. Commercially available polyether amines include the polyether polyamines (available under the trade designation “JEFFAMINE” from Huntsman Corporation and 4,7, 10-trioaxatridencane-1,13-diamine (TTD) (available from BASF). A preferred latent amine curing agent is Dyhard 100E from AlzChem.

Preferred other curatives whose performance can be accelerated by the urones of this invention are amino sulfones such as 4,4 diaminodiphenyl sulfone (sometimes known as 4,4-DDS) and 3,3 diaminodiphenyl sulfone (sometimes known as 3,3-DDS) which are typically used as curatives in resin formulations used in the production of aerospace components.

When the urones of this invention are used as accelerators for other curatives in epoxy resins we prefer to use from 0.1 to 5 wt % more preferably 0.2 to 2 wt % of the urone based on the weight of epoxy resin.

In a further embodiment of the invention there is provided a resin formulation comprising the urone of the invention in combination with a curing agent and at least one resin component such as an epoxy, polyisocyante and a phenolic resin particularly an epoxy resin. The resin formulation is preferably in the form of a one-component resin formulation which does not require any further mixing of components before its use.

In a further embodiment the invention provides the use of a resin formulation of this invention as a matrix in fibre reinforced composites which may be a prepreg or may be obtained by resin infusion with the resin formulation of dry fibrous material laid up in a mould. The invention further provides a fibre reinforced composite obtained by the thermal curing of such a resin matrix. This can be accomplished in any conventional manner preferred methods being in a press or in a vacuum bag.

In another embodiment of the invention there is provided a moulding material comprising a thermosetting resin formulation of this invention in combination with a fibrous reinforcement material. The fibrous reinforcement material may be provided: as a woven fabric or a multi-axial fabric to form a prepreg, as individual fibre tows for impregnation with the resin composition to form towpregs, or as chopped fibres, short fibres or filaments to form a moulding compound. The preferred fibrous material is selected from carbon fibre, glass fibre, aramid and mixtures thereof. The moulding material may be constructed from a cast resin film which contains the resin formulation and which is combined with a fibrous reinforcement layer. Preferably the resin film impregnates the fibrous reinforcement which may be accomplished by pressing a layer of resin onto the fibrous material or by infusion of the resin into the fibrous material within a mould.

In a further embodiment of the invention there is provided an adhesive comprising a resin formulation of this invention in combination with at least one filler.

The compositions of this invention are storage stable at ambient temperature prior to curing and are capable of fast curing whilst the Tg, the retained Tg and the mechanical properties of the cured resin enable use of the cured resin formulation in industrial structural applications particularly as fibre reinforced materials which are useful as automotive and aerospace structural components as well as sporting goods and wind turbine components.

Where the resin is an epoxy resin it may be mono-functional or multifunctional, preferably at least difunctional. In an embodiment, the epoxy resin component (A) may be selected from various conventionally-known polyepoxy compounds. Examples thereof include: aromatic glycidyl ether compounds such as bis(4-hydroxyphenyl)propane diglycidyl ether, bis(4-hydroxy-3,5-dibromophenyl)propane diglycidyl ether, bis(4-hydroxyphenyl)ethane diglycidyl ether, bis(4-hydroxyphenyl)methane diglycidyl ether, resorcinol diglycidyl ether, phloroglucinol triglycidyl ether, trihydroxy biphenyl triglycidyl ether, tetraglycidyl benzophenone, bisresorcinol tetraglycidyl ether, tetramethyl bisphenol A diglycidyl ether, bisphenol C diglycidyl ether, bisphenol hexafluoropropane diglycidyl ether, 1,3-bis[1-(2,3-epoxypropoxy)-1-trifluoromethyl-2,2,2-trifluoroethyl]benzene, 1,4-bis[1-(2,3-epoxypropoxy)-1-trifluoromethyl-2,2,2-trifluoromethyl]benzene, 4,4′-bis(2,3-epoxypropoxy)octafluorobiphenyl, and phenolic novolac type bisepoxy compounds; alicyclic polyepoxy compounds such as alicyclic diepoxy acetal, alicyclic diepoxy adipate, alicyclic diepoxy carboxylate, and vinylcyclohexene dioxide; glycidyl ester compounds such as diglycidyl phthalate, diglycidyl tetrahydrophthalate, diglycidyl hexahydrophthalate, dimethylglycidyl phthalate, dimethylglycidyl hexahydrophthalate, diglycidyl-p-oxybenzoate, diglycidylcyclopentane-1,3-dicarboxylate, and dimer acid glycidyl ester; glycidyl amine compounds such as diglycidyl aniline, diglycidyl toluidine, triglycidyl aminophenol, tetraglycidyl diaminodiphenyl methane, and diglycidyl tribromoaniline; and heterocyclic epoxy compounds such as diglycidylhydantoin, glycidyl glycidoxyalkylhydantoin, and triglycidyl isocyanurate; and oligomer compounds thereof.

Examples of the liquid epoxy resin include polyalkylene ether type epoxy compounds such as (poly)ethylene glycol diglycidyl ether, (poly)propylene glycol diglycidyl ether, and trimethylolpropane triglycidyl ether; glycidyl ester type epoxy compounds such as dimer acid diglycidyl ester, phthalic acid diglycidyl ester, and tetrahydrophtalic acid diglycidyl ester; and homopolymers of glycidyl (meth)acrylate, allyl glycidyl ether and the like or copolymers of these monomers with other soft unsaturated monomers. In this context, soft unsaturated monomer refers to a monomer which contains a homopolymer which has a glass transition temperature of less than 60° C. Examples of soft unsaturated monomers include methyl acrylate, ethyl acrylate, butyl(meth)acrylate, isobutyl(meth)acrylate, 2-ethylhexyl(meth)acrylate, and lauryl methacrylate.

A liquid curable epoxy resin composition of the present invention is particularly useful as a one-component liquid epoxy resin prepreg matrix resin formulation which is excellent in both storage stability, and increased latency similar to the latency of comparable unaccelerated formulations. The resins also have good curing characteristics and provide a cured product having excellent characteristics, particularly organic solvent resistance.

When the epoxy resin composition of the present invention is used as a prepreg resin formulation, additives such as fillers, viscosity modifiers, tougheners, pigments, thixotropic agents, and fire retardants, or the like can be optionally mixed into the formulation to enhance its mechanical performance and flow behaviour during cure.

The compositions of this invention may include other typical additives used in thermosetting resin formulations such as impact modifiers, fillers, antioxidants and the like.

The amount of the urones of this invention that is used in the resin formulation depends upon the required function of the compound and depending upon whether it is used as the primary or sole curative or as an accelerator for another curative. The amount can range from 0.01 to 20 parts of the urone of this invention or mixtures thereof per 100 parts of resin, preferably from 0.1 to 15 parts, preferably from 1 to 15 parts and most particularly preferably from 2 to 15 parts per 100 parts of resin. Also preferred are amounts in which there are used, per 100 parts of resin, from 1 to 12 parts, in particular from 2 to 12 parts, more preferably from 3 to 12 parts, particularly preferably from 4 to 12 parts and most particularly preferably from 5 to 12 parts of the urone of the invention.

With these amounts it is possible to generate, at a temperature of from 60 to 180° C., a maximum heat flow in the epoxy resin composition of from 0.05 to 0.99 W/g (based on the mass of epoxy resin), so that the epoxy resin composition cures fully.

We have found that when the urones of this invention are used as accelerators for other curatives higher curing temperature such as from 150 to 190° C. may be used. We have also found that the time required for the viscosity of the resin formulation to double at certain temperatures below the curing temperature is increased thereby indicating the improvement in the latency and storage stability of the formulation compared to formulations containing dimethylamine blocked urones and similar latency to unaccelerated formulations.

An epoxy resin composition is considered to be fully cured if the epoxy resin composition cures to the extent of 80%, preferably 90%, more preferably 95%, yet more preferably 98%, in particular 99% and most preferably 100%. Accordingly, the epoxy groups in the cured epoxy resin composition have reacted to the extent of in particular 80%, preferably 90%, more preferably 95%, yet more preferably 98%, in particular 99% and most preferably 100%.

The compositions of this invention can contain other components conventionally used in epoxy resin formulations such as impact modifiers and fillers as set out below.

### Impact Modifiers

The composition may comprise an impact modifier. Impact modifiers are widely used to improve the impact strength of cured resin compositions with the aim to compensate for their inherent brittleness and crack propagation. Impact modifiers may comprise rubber particles such as CTBN rubbers (carboxyl-terminated butadiene-acrylonitrile) or core shell particles which contain a rubber or other elastomeric compound encased in a polymer shell. The advantage of core shell particles over rubber particles is that they have a controlled particle size of the rubber core for effective toughening and the grafted polymer shell ensures adhesion and compatibility with the epoxy resin composition. Examples of such core shell rubbers are disclosed in EP0985692 and in WO 2014062531.

Alternative impact modifiers include methylacrylate based polymers, polyamides, acrylics, polyacrylates, acrylate copolymers, phenoxy based polymers, and polyethersulphones.

### Fillers

In addition the composition may comprise one or more fillers to enhance the flow properties 35 of the composition. Suitable fillers may comprise talc, microballoons, flock, glass beads, silica, fumed silica, carbon black, fibres, filaments and recycled derivatives, and titanium dioxide.

A prepreg resin formulation of the present invention can be prepared by uniformly mixing the curative system of the invention, the resin and other additives using a pot mill, a ball mill, a bead mill, a roll mill, a homogenizer, Supermill, Homodisper, a universal mixer, Banbury mixer, a kneader, or the like.

Since a prepreg resin formulation of the present invention can be a one-component type that has both high storage stability and excellent thermosetting properties, it can be suitably used for applications which require long term storage or storage in unconditioned facilities at room temperature.

The resin formulations of this invention are particularly useful as the curable matrix for the production of fibre reinforced articles such as in prepregs that are used for the production of components for wind turbines and components for the aerospace industry such as aircraft wings and fuselages. The formulations may also be used in the production of automobile components and components used in shipping and in the production of sporting goods such as skis.

The invention is illustrated by the following examples in which Examples 1 to 5 show the preparation of urones used in this invention and Examples 6 and 7 show the use of the urones of Examples 1 to 4 as accelerators for the curing of the epoxy resins and compares their performance with other urones.

The following test methods were used.

The cured Tg of a resin is measured in accordance with ASTM D7028 (Glass Transition Temperature (DMA Tg) of Polymer Matrix Composites by Dynamic Mechanical Analysis (DMA)).

The heat released during the curing reaction is related to the total heat for fully curing and can be measured using Digital Scanning Calorimetry as follows. A reference resin sample is heated from 10° C. to 350° C. at 10° C./min rate to full cure (100%) and the generated heat ΔHi is recorded. The degree of cure of a particular resin sample of the same composition as the reference resin sample can then be measured by curing the sample to the desired temperature and at the desired rate and for the desired time by heating the sample at these conditions and measuring the heat ΔHe generated by this cure reaction. The degree of cure (Cure %) is then defined by:

Cure %=[(ΔHi−ΔHe)/ΔHi]×100[%]

where ΔHi is the heat generated by the uncured resin heated from 10° C. up to fully cured at 350° C. and ΔHe the heat generated by the certain degree cured resin heated up to a desired temperature and rate.

Dynamic mechanical analysis (DMA) was performed using a Q800 instrument on cured resin to determine glass transition temperatures at a heating rate of 5° C./min and at a frequency of 1 Hz, and at an amplitude of 30 μm.

Dynamic and Isothermal rheology was performed in oscillation mode using a strain of 10% and a frequency of 3 HZ using a MCR92 from Anton Paar. Dynamic rheology was performed with a heating rate of 2° C./min.

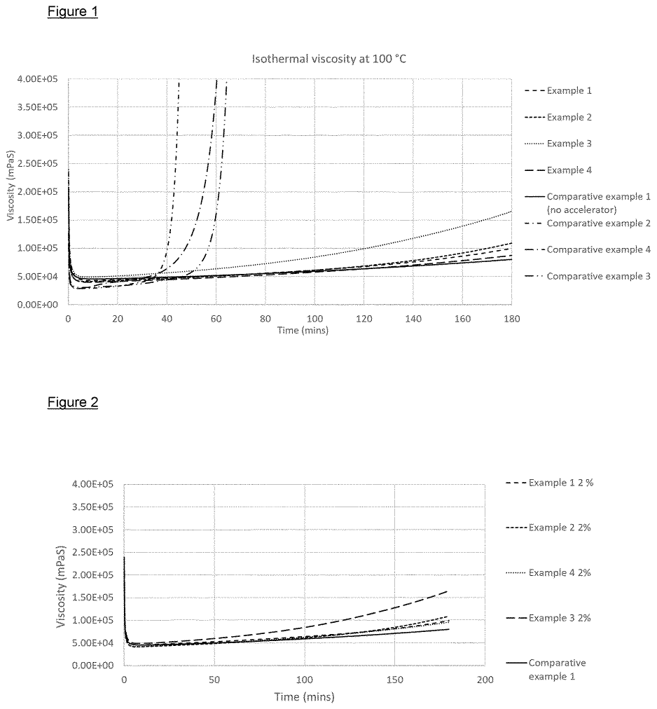

The structures of the compounds produced in Examples 1 to 5 was confirmed by H-NMR and the results of the tests employed in Examples 6 and 7 are shown in FIGS. 1 and 2.

### EXAMPLE 1

### Preparation of N,N-bis(2-hydroxyphenyl)-1,4-piperazinedicarboxadmide, OHPPDA

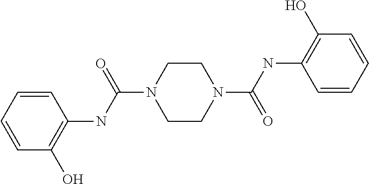 [SMILES: O=C(Nc1ccccc1O)N1CCN(C(=O)Nc2ccccc2O)CC1]

To a 250 ml round bottom flask was added 50.0 g of 2-benzoxazolinone and 16.7 g of piperazine. The flask was purged with nitrogen and 450 ml of 2-propanol was added. The solution was heated to reflux and stirred at this temperature for at least 14 hours before cooling to room temperature. The precipitate was collected by vacuum filtration and washed with 2-proponal before drying in a vacuum oven at 60° C. overnight to give 43.2 g of an off-white powder, with an endothermic decomposition temperature of 206° C. The yield was 66.3%.

¹H NMR (DMSO-d6): δ (ppm) 9.68 (s, 2H, OH); 8.02 (s, 2H, NH); 7.43 (dd, 2H, Ar H); 6.92 (td, 2H, Ar H); 6.84 (dd, 2H, Ar H); 6.76 (td, 2H, Ar H); 3.54 (s, 8H, CH₂);

### EXAMPLE 2

### Preparation of N,N-bis(5-chloro-2-hydroxyphenyl)-1,4-piperazinedicarboxadmide, COHPPDA

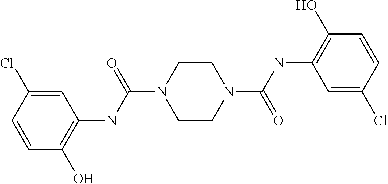 [SMILES: O=C(Nc1cc(Cl)ccc1O)N1CCN(C(=O)Nc2cc(Cl)ccc2O)CC1]

The urone was prepared using 25.0 g of 6-nitro-1,3-benzoxazolinone, 6.4 g of piperazine and 120 ml of 2-propanol in the same procedure set out in example 1 using a reflux time of 12.5 hours to give 23.8 g of an off-white powder, with an endothermic decomposition temperature of 239° C. and a 76.0% yield.

¹H NMR (DMSO-d6): δ (ppm) 9.78 (bs, 2H, OH); 7.93 (s, 2H, NH); 7.65 (d, 2H, Ar H); 6.92 (dd, 2H, Ar H); 6.84 (d, 2H, Ar H); 3.52 (s, 8H, CH₂);

### EXAMPLE 3

### Preparation of N,N-bis(4-nitro-2-hydroxyphenyl)-1,4-piperazinedicarboxamide, 4-NOHPPDA

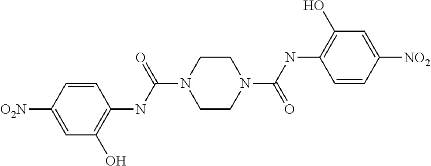 [SMILES: O=C(Nc1ccc([N+](=O)[O-])cc1O)N1CCN(C(=O)Nc2ccc([N+](=O)[O-])cc2O)CC1]

To a 250 ml round bottom flask was added 10.3 g of 6-nitro-1,3-benzoxazolinone and 30 ml of dimethylacetamide. The solution was heated to 100° C. and a solution of 2.4 g of piperazine and 5.9 g of triethylamine in 30 ml of dimethylacetamide, was added dropwise. The mixture was stirred at 100° C. for 2.5 hours before cooling to room temperature. The reaction mixture was poured into a 100 ml of rapidly stirred dilute hydrochloric acid. The precipitate was collected by filtration and washed with water then acetone before drying in a vacuum oven at 60° C. overnight to give 7.2 g of a yellow solid, with an exothermic decomposition commencing at 273° C. The yield was 56.3%.

1H NMR (DMSO-d6): δ (ppm) 10.96 (s, 2H, OH); 8.13 (s, 2H, NH); 8.02 (d, 2H, Ar H); 7.72 (dd, 2H, Ar H); 7.66 (d, 2H, Ar H); 3.57 (s, 8H, CH2);

### EXAMPLE 4

### Preparation of N,N-bis(5-nitro-2-hydroxyphenyl)-1,4-piperazinedicarboxamide, 5-NOHPPDA

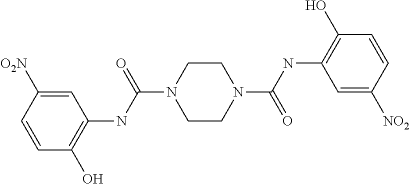 [SMILES: O=C(Nc1cc([N+](=O)[O-])ccc1O)N1CCN(C(=O)Nc2cc([N+](=O)[O-])ccc2O)CC1]

The urone was prepared using 12.5 g of 5-nitro-1,3-benzoxazolinone, 2.9 g of piperazine, 13.4 ml of triethylamine and 70 ml of dimethylacetamide, the procedure of Example 3, was used with a reaction time of 12 hours to give 13.2 g of a pale yellow powder, with an exothermic decomposition commencing at 292° C. The yield was 84.1%.

1H NMR (DMSO-d6): δ (ppm) 11.42 (s, 2H, OH); 8.60 (s, 2H, Ar H); 8.07 (d, 2H, NH); 7.85 (dd, 2H, Ar H); 6.98 (d, 2H, Ar H); 3.56 (s, 8H, CH2);

### EXAMPLE 5

### Preparation of an Oligomer from Reaction of Piperazine and 2,2-bis(3H-1,3-benzoxazol-2-one)sulfone polySBOHPPDA

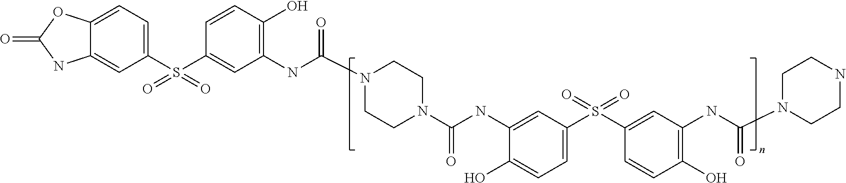

To a 20 ml sample vial was added 1.07 g of 2,2-bis(3H-1,3-benzoxazol-2-one)sulfone, 0.27 g of piperazine and 10 ml of dimethylacetamide. The sealed vial was heated on a hot plate set at 115° C. for 48 hours. After cooling to room temperature, the reaction mixture was poured into a stirred solution of dilute hydrochloric acid. The precipitate was collected by filtration and washed with water before drying in a vacuum oven at 60° C. overnight to give 0.78 g of a cream powder, with two endothermic decomposition temperatures of 201° C. and 262° C. 1H NMR analysis in DMSO-d6 showed an n of ~5.5. The yield was 58.2%.

### EXAMPLE 6

The urones of Examples 1-4 were used as accelerators for a 4,4-DDS curing system, the composition of which is shown in Table 1.

TABLE 1

| Component | Composition (wt. %) |
|---|---|
| MY0610 | 27.64 |
| LY1556 | 24.45 |
| 5003P | 15.00 |
| Polyamide particle | 13.50 |
| 4,4-DDS | 18.40 |
| Urone | 1.00 |

MY0610 is an epoxy resin based on triglycidyl-m-aminophenol
4,4-DDS is 4,4′-diphenyldiaminosulphone from Huntsman Advanced Materials
LY1556 an Araldite Epoxy resin from Huntsman
5003P is a polyethersulfone thermoplastic supplied by Sumitomo Chemical
polyamide particle supplied by Arkema as Orgasol

The performance of the materials as cure accelerators were compared to the following comparative formulations containing no urone or urones of the prior art.

| Comparative | Urone |
|---|---|
| Example | Accelerator |
| 1 | None |
| 2 | UR500 |
| 3 | U52 |
| 4 | OHFU |

### Where

UR500 is (3,3¹-(4-methyl-1,3 phenylene) bis (1,1 dimethyl urea) as in U.S. Pat. No. 9,633,609
U52 is (4,41 methylene diphenylene bis(N,N dimethyl urea) from Huntsman advanced Materials Company
OHFU is Orthohydroxyfenuron prepared as described in U.S. Pat. No. 4,404,356

The mixtures were cured in a fan oven at 180° C. Tables 2 and 3 show the % cure and the Tg of the formulations after 45, 75 and 120 minutes cure at 180° C., respectively. The tables show formulations containing the urones from Examples 1-4 can achieve improved % cure under a reduced cure cycle time compared to the unaccelerated formulation, whilst at the same time having improved (higher) Tgs compared to comparative Examples 2-4, which are accelerated with prior urone accelerators.

TABLE 2

% cure after curing at 180° C. for

| Urone | 45 minutes | 75 minutes | 120 minutes |
|---|---|---|---|
| Example 1 | 92.2 | 95.5 | 96.8 |
| Example 2 | 93.6 | 95.8 | 97.1 |
| Example 3 | 92.6 | 96.5 | 98.4 |
| Example 4 | 89.4 | 95.9 | 97.5 |
| Comparative | 81.1 | 85.1 | 91.8 |
| example 1 | | | |
| Comparative | 95.9 | n/a | 97.5 |
| example 2 | | | |
| Comparative | 94.6 | n/a | 98.5 |
| example 3 | | | |
| Comparative | 96.1 | n/a | 98.4 |
| example 4 | | | |

TABLE 3

Tg after curing at 180° C. for

| Urone | 45 minutes | 75 minutes | 120 minutes |
|---|---|---|---|
| Example 1 | 156 | 170 | 171 |
| Example 2 | 158 | 168 | 173 |
| Example 3 | 161 | 173 | 184 |
| Example 4 | 154 | 171 | 173 |
| Comparative | 111 | 121 | n/a |
| example 1 | | | |
| Comparative | 130 | n/a | 136 |
| example 2 | | | |
| Comparative | 131 | n/a | 143 |
| example 3 | | | |
| Comparative | 138 | n/a | 145 |
| example 4 | | | |

The time required for the viscosity of the formulations to double when heated to 80° C. and 100° C. was found to be

Time to double viscosity (mins) at

| Urone | 80° C. | 100° C. |
|---|---|---|
| Example 1 | >180 | 159.8 |
| Example 2 | >180 | 158.0 |
| Example 3 | >180 | 175.0 |
| Example 4 | >180 | 163.3 |
| Comparative | >180 | 176.7 |
| example 1 | | |
| Comparative | 144.3 | 36.7 |
| example 2 | | |
| Comparative | >180 | 51.2 |
| example 3 | | |
| Comparative | 166.7 | 37.3 |
| example 4 | | |

These results are also shown in the graphs of FIG. 1 which shows the isothermal viscosity at 100° C. for the formulations containing accelerators from Examples 1-4, compared to the comparative Examples 1-4 formulations. The results show that the formulations accelerated with urones from Examples 1-4 have excellent latency at elevated temperatures with little difference in viscosity compared to that of the unaccelerated formulation, comparative Example 1, over 3 hours.

### EXAMPLE 7

The composition of the formulations of Example 6 was modified by increasing the content of urone as shown in Table 4. Tables 5 and 6 show the % cure and the Tg of these formulations after 45, 75 and 120 minutes at 180° C., respectively. The increase in loading of the urone leads to a further improvement in % cure, particularly under the reduced cure cycle time of 45 minutes. The Tg achieved after the 45-minute cure cycle was also improved compared to the formulations containing 1 wt. % of the accelerators as in Examples 1-4.

TABLE 4

| Component | Composition (wt. %) |
|---|---|
| MY0610 | 27.25 |
| LY1556 | 24.11 |
| 5003P | 15.00 |
| Polyamide particle | 13.50 |
| 4,4-DDS | 18.14 |
| Urone | 2.00 |

TABLE 5

% cure after curing at 180° C. for

| Urone | 45 minutes | 75 minutes | 120 minutes |
|---|---|---|---|
| Example 1 | 97.0 | 96.5 | 98.4 |
| Example 2 | 97.5 | 98.6 | 98.3 |
| Example 3 | 97.4 | 98.9 | 99.7 |
| Example 4 | 96.0 | 97.0 | 99.2 |

TABLE 6

Tg after curing at 180° C. for

| Urone | 45 minutes | 75 minutes | 120 minutes |
|---|---|---|---|
| Example 1 | 159 | 165 | 163 |
| Example 2 | 163 | 168 | 168 |
| Example 3 | 165 | 169 | 179 |
| Example 4 | 167 | 173 | 177 |

The time required for the viscosity of the formulations to double when heated to 80° C. and 100° C. was found to be

Time to double viscosity (mins) at

| Urone | 80° C. | 100° C. |
|---|---|---|
| Example 1 | >180 | 168.3 |
| Example 2 | >180 | 147.3 |
| Example 3 | >180 | 118.5 |
| Example 4 | >180 | 157.0 |
| Comparative | >180 | 176.7 |
| example 1 | | |

These results are shown in the graph of FIG. 2 hereto which shows the isothermal viscosity at 100° C. of the formulations of Example 7 containing the urone accelerators at 2 wt. %. The graph shows that although the loading of the accelerators is increased from Examples 1-4, this does not have a significant effect on the latency of the formulation over 3 hours at elevated temperatures, compared to the formulations containing 1 wt. % of the accelerators from Examples 1-4, and in some instances the unaccelerated formulation.

1. (canceled)

2. An accelerator for the curing of resins comprising a urone compound containing at least 2 orthohydroxyphenyl urea according selected from one or more compounds of the following formulae 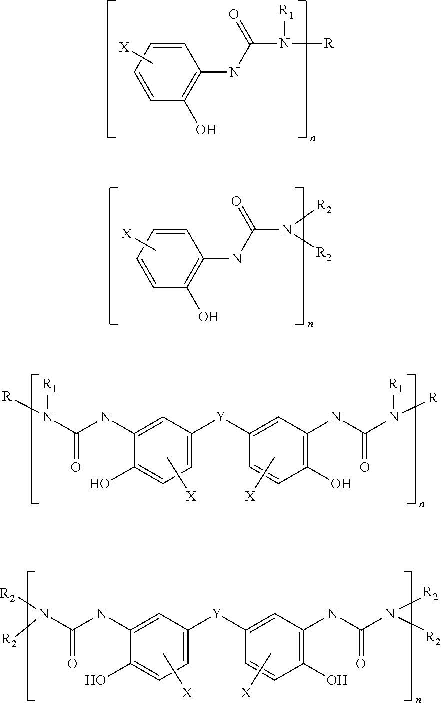
where R is a divalent or multivalent linear or branched aliphatic hydrocarbon radical (substituted or unsubstituted), a divalent or multivalent hetero interrupted aliphatic hydrocarbon radical (substituted or unsubstituted), a divalent or multivalent aromatic hydrocarbon interrupted aliphatic hydrocarbon radical (substituted or unsubstituted);
where R₁ is at each occurrence, independently of one or another, selected from H, linear or branched monovalent aliphatic radical (substituted or unsubstituted), or an unsubstituted, halo substituted and/or alkyl substituted monovalent aromatic radical;
where both R₂ groups together forms a divalent or multivalent organic cycloaliphatic hydrocarbon radical (substituted or unsubstituted), a divalent or multivalent hetero interrupted cycloaliphatic hydrocarbon radical (substituted or unsubstituted), a divalent or multivalent aromatic hydrocarbon interrupted cycloaliphatic hydrocarbon radical (substituted or unsubstituted);
where Y is S, SO2, SO, PR₂, NR₂, P═O(OX or OH), C═O, a linear or branched aliphatic radical (substituted or unsubstituted), or an unsubstituted, halo substituted and/or alkyl substituted aromatic radical;
where n is at least 2; and
where X is at each occurrence, independently of one or another, selected from H, OH, NH₂, NO₂, nitrile, a halogen, a substituted or unsubstituted linear or branched aliphatic radical, and a substituted or unsubstituted aromatic radical.

3. The accelerator according to claim 2 in which Y, is substituted with further orthohydroxyphenylurone functionalities units, to form a tri or tetravalent bridging unit.

4. (canceled)

5. The composition comprising a urone compound of claim 2 in combination with at least one resin component selected from an epoxy, a polyisocyanate and a phenolic resin.

6. (canceled)

7. (canceled)

8. composition according to claim 5 in which curing agent is selected from ethylenediamine, diethylenediamine, diethylenetriamine, triethylenetetramine, propylene diamine, tetraethylenepentamine, hexaethyleneheptamine, hexamethylenediamine, cyanoguanidine, 2-methyl-1,5-pentamethylene-diamine, 4,7,10-trioxatridecan-1,13-diamine, aminoethylpiperazine, dicyanopolyamides, 4,4′-diaminodiphenyl sulfone or 3,3′-diaminodiphenyl sulfone, bisanilines, dihydrazides, polyamines, polyether amines having one or more amine moieties, and mixtures thereof.

9. The moulding material comprising a composition according to claim 5 in combination with a fibrous reinforcement material.

10. (canceled)

11. The moulding material according to claim 9 in which the fibrous material is selected from carbon fibre, glass fibre and aramid.

12. (canceled)

13. (canceled)

14. (canceled)

15. (canceled)

16. (canceled)

17. (canceled)

18. (canceled)

19. (canceled)

20. (canceled)

21. (canceled)

22. (canceled)
